(19) United States
(12) Patent Application Publication
(10) Pub. No.: US 2016/0244453 A1
(43) Pub. Date: Aug. 25, 2016

(54) TREATMENT OF VIRAL AND INFECTIOUS DISEASES USING AN INHIBITOR OF CBP/CATENIN

(71) Applicant: PRISM PHARMA CO., LTD., Kanagawa (JP)

(72) Inventors: Hiroyuki KOUJI, Kanagawa (JP); Takenao ODAGAMI, Kanagawa (JP); Howard DITTRICH, La Jolla, CA (US)

(73) Assignee: PRISM Pharma Co., Ltd., Kanagawa (JP)

(21) Appl. No.: 14/436,670

(22) PCT Filed: Oct. 21, 2013
(86) PCT No.: PCT/JP2013/079054

§ 371 Date: Apr. 17, 2015

(51) Int. Cl. C07D 487/04; A61K 31/675; A61K 31/53; A61K 45/06; C07F 9/6561

(52) U.S. Cl. CPC C07D 487/04; A61K 45/06; C07F 9/6561; A61K 31/53; A61K 31/675

(57) ABSTRACT

The present disclosure relates generally to alpha-helix mimetic structures and specifically to alpha-helix mimetic structures that are inhibitors of β-catenin. The disclosure also relates to applications in the treatment of viral and infectious diseases, including infection by HIV, HPV, HBV, HSV, and bacteria including Mycobacterium, Shigella, and Listeria, and pharmaceutical compositions comprising such alpha helix mimetic β-catenin inhibitors.

### CROSS-REFERENCE TO RELATED APPLICATIONS

This application claims priority to U.S. provisional application 61/716,116, filed Oct. 19, 2012; U.S. provisional application 61/748,621, filed Jan. 3, 2013; and U.S. provisional application 61/820,995, filed May 8, 2013; each of which is incorporated herein in its entirety.

### BACKGROUND OF THE DISCLOSURE

Wnt/β-catenin signaling is emerging as a forerunner for its critical roles in many facets of human biology. This signaling pathway has roles in embryogenesis, organogenesis, and maintaining tissue and organ homeostasis. However, aberrant activation of this pathway is also evident in many viral and infectious disease conditions.

Human immunodeficiency virus (HIV) is a lentivirus (a member of the retrovirus family) that causes acquired immunodeficiency syndrome (AIDS), a condition in humans in which progressive failure of the immune system allows life-threatening opportunistic infections and cancers to thrive.

Human papillomavirus (HPV) is a virus from the papillomavirus family that is capable of infecting humans. Like all papillomaviruses, HPVs establish productive infections only in keratinocytes of the skin or mucous membranes. While the majority of the known types of HPV cause no symptoms in most people, some types can cause warts (verrucae), while others can—in a minority of cases—lead to cancers of the cervix, vulva, vagina, penis, oropharynx and anus.

Herpes simplex virus 1 and 2 (HSV-1 and HSV-2), also known as Human herpes virus 1 and 2 (HHV-1 and -2), are two members of the herpes virus family, Herpesviridae, that infect humans. Both HSV-1 (which produces most cold sores) and HSV-2 (which produces most genital herpes) are ubiquitous and contagious. They can be spread when an infected person is producing and shedding the virus. Symptoms of herpes simplex virus infection include watery blisters in the skin or mucous membranes of the mouth, lips or genitals.

Tuberculosis, MTB, or TB (short for tubercle bacillus) is a common, and in many cases lethal, infectious disease caused by various strains of mycobacteria, usually Mycobacterium tuberculosis. Tuberculosis typically attacks the lungs but can also affect other parts of the body. It is spread through the air when people who have an active TB infection cough, sneeze, or otherwise transmit their saliva through the air.

Additional microorganisms causing intracellular infections include Listeria monocytogenes (which causes listeriosis and septicemia) and Shigella species (which cause dysentery). These bacteria are significant causes of food-borne illness and neonatal/childhood infection and mortality. Listeria is a virulent food-borne pathogen that infects multiple cell types including phagocytic cells such as macrophages. L. monocytogenes kills 20 to 30% of individuals with clinical infections, and is a leading cause of meningitis in newborns. Shigella species, particularly S. flexneri, S. sonnei, and S. dysenteriae, infect cells of the gastrointestinal tract and cause severe gastrointestinal symptoms such as diarrhea and stomach cramps. Shigella infections are responsible for over 90 million cases of dysentery and over 100,000 deaths each year, mostly in children in developing countries.

A cellular response to intracellular infection is autophagy, a process by which cells encapsulate and destroy foreign and unwanted intracellular components. Autophagy begins with encapsulation of the microorganism in an intracellular endosome/autophagosome. The autophagosome fuses with a lysosome containing lytic enzymes to form an autolysosome. Inside the autolysosome, the acidic and lytic environment kills the microorganism. Various microorganisms, such as Mycobacterium tuberculosis and HIV, circumvent this process by downregulating or inhibiting autophagy and maintaining intracellular infection (Specter, Topics Antiviral Med. 19:6-10, 2011.)

Wnt signaling is involved in the immune response on multiple levels. Wnt signaling is involved in regulation of T-cell development, and also regulates autophagy. For example, decreased levels of β-catenin have been shown to up-regulate autophagy (Nguyen, et al., J. Cell. Mol. Med. 13:3687-3698, 2009). Thus, Wnt signaling plays several potential roles in response to microorganism infection.

A study by Gattinoni et al. (Nat Med. 15(7): 808-813, 2009) revealed a key role for Wnt signaling in the maintenance of stemness in mature memory CD8⁺ T cells. The results of this study have important implications for the design of novel vaccination strategies and adoptive immunotherapies by targeting the Wnt pathway.

A study by Bulut et al. (PLoS One 2011; 6(11): e27243) identified a potential link between activation of the Wnt signaling pathway and its contribution to HPV-mediated cervical cancer. These results indicate that activation of the canonical Wnt pathway might represent secondary events that are required for malignant transformation of HPV-infected epithelial cells. Targeting the canonical Wnt pathway may therefore provide the basis for developing clinical interventions to prevent disease progression in populations at risk for HPV infection and to treat advanced cervical cancers, as well as other viral and infectious diseases.

### BRIEF SUMMARY OF THE DISCLOSURE

This disclosure presents methods of treating infectious diseases, including infection by HIV, HPV, HBV, HSV, and bacteria including Mycobacterium, Shigella, and Listeria, by administration of an inhibitor of β-catenin signaling, alone or in combination with additional antiviral or antibacterial treatments. This disclosure also provides alpha helix mimetic β-catenin inhibitor compounds, and compositions comprising an inhibitor of β-catenin.

### BRIEF DESCRIPTION OF THE DRAWINGS

FIG. 1: HIV replication (ng/ml of HIV p24 antigen) in macrophages showing dose response (reduced amounts of p24 antigen) with increasing concentrations of Compound C.

FIGS. 2A-2B: Compound A at 1 and 2 μM induces autophagy. (A-B), Treatment of cells in the presence or absence of Compound A (Cmpd A) or rapamycin (Rapa, a positive control for autophagy) shows increased amount of LC3B II and decreased amount of LC3B I relative to negative control (no Compound A, no rapamycin), an indicator of autophagy. At 2 μM Compound A, and with rapamycin, levels of both LC3B II and LC3B I are greatly reduced relative to negative control. (B), Sequestome 1/p62 (SQSTM1) levels are reduced in rapamycin and Compound A-treated cells relative to negative control, indicating increased autophagy.

FIGS. 3A-3B: (A), Pie charts reflecting the proportion of indicated T cell subsets after 10-day incubation of sorted Tscm (left panel) or Tcm (right panel) in the presence or absence of the β-catenin inhibitor Compound C. Results from one representative study subject are shown. (B), Proportion of sorted CD4 Tscm or CD4 Tcm who maintain their original CCR7+ CD62L+ phenotype after 10 day incubation with or without Compound C. Cumulative data from n=3 three study subjects are shown.

### DETAILED DESCRIPTION OF THE DISCLOSURE

Recently, non-peptide compounds have been developed which mimic the secondary structure of reverse-turns found in biologically active proteins or peptides. For example, U.S. Pat. No. 5,440,013 and published PCT Applications Nos. WO94/03494, WO01/00210A1, and WO01/16135A2 each disclose conformationally constrained, non-peptidic compounds, which mimic the three-dimensional structure of reverse-turns. In addition, U.S. Pat. No. 5,929,237 and its continuation-in-part U.S. Pat. No. 6,013,458, disclose conformationally constrained compounds which mimic the secondary structure of reverse-turn regions of biologically active peptides and proteins. In relation to reverse-turn mimetics, conformationally constrained compounds have been disclosed which mimic the secondary structure of alpha-helix regions of biologically active peptide and proteins in WO2007/056513 and WO2007/056593.

This disclosure provides novel compounds, pharmaceutical compositions and methods of treatment for viral and infectious diseases, including infection by HIV, HPV, HBV, HSV, and bacteria including Mycobacterium, Shigella, and Listeria. The inventors have determined that inhibiting β-catenin signaling is an effective approach to the treatment of these diseases.

The structures and compounds of the alpha helix mimetic β-catenin inhibitors of this invention are disclosed in WO 2010/044485, WO 2010/128685, WO 2009/148192, and US 2011/0092459, each of which is incorporated herein by reference in its entirety. These compounds have now been found to be useful in the treatment of viral and infectious diseases, including HIV, HPV, HBV, HSV, and bacteria including Mycobacterium, Shigella, and Listeria.

The preferable structure of the alpha helix mimetic β-catenin inhibitors of this invention have the following formula (I):

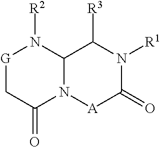

wherein

### A is —CHR⁷—,

wherein

R⁷ is optionally substituted arylalkyl, optionally substituted heteroarylalkyl, optionally substituted cycloalkylalkyl or optionally substituted heterocycloalkylalkyl;

### G is —NH—, —NR⁶—, or —O—

wherein

R⁶ is lower alkyl or lower alkenyl;

R¹ is —Ra—R¹⁰;

wherein

Ra is optionally substituted lower alkylene and

R¹⁰ is optionally substituted bicyclic fused aryl or optionally substituted bicyclic fused heteroaryl;

R² is —(CO)—NH—Rb—R²⁰,

wherein

Rb is bond or optionally substituted lower alkylene; and

R²⁰ is optionally substituted aryl or optionally substituted heteroaryl; and

R³ is C₁₋₄ alkyl.
These compounds are especially useful in the prevention and/or treatment of viral and infectious diseases, including HIV, HPV, HBV, HSV, and tuberculosis.

The more preferable structure of the alpha helix mimetic β-catenin inhibitors of this invention have the following substituents in the above-mentioned formula (I):

### A is —CHR⁷—,

wherein

R⁷ is arylalkyl optionally substituted with hydroxyl or C₁₋₄ alkyl;

### G is —NH—, —NR⁶—, or —O—

wherein

R⁶ is C₁₋₄ alkyl or C₁₋₄ alkenyl;

R¹ is —Ra—R¹⁰;

wherein

Ra is C₁₋₄ alkylene and

R¹⁰ is bicyclic fused aryl or bicyclic fused heteroaryl, optionally substituted with halogen or amino;

R² is —(CO)—NH—Rb—R²⁰,

wherein

Rb is bond or C₁₋₄ alkylene; and

R²⁰ is aryl or heteroaryl; and

R³ is C₁₋₄ alkyl.
These compounds are especially useful in the prevention and/or treatment of viral and infectious diseases, including HIV, HPV, HBV, HSV, and bacteria including Mycobacterium, Shigella, and Listeria.

The most preferable alpha helix mimetic β-catenin inhibitors of this invention are as follows:
(6S,9S)—N-benzyl-6-(4-hydroxybenzyl)-2,9-dimethyl-8-(naphthalen-1-ylmethyl)-4,7-dioxooctahydro-1H-pyrazino[2,1-c][1,2,4]triazine-1-carboxamide,
(6S,9S)-2-allyl-N-benzyl-6-(4-hydroxybenzyl)-9-methyl-8-(naphthalen-1-ylmethyl)-4,7-dioxooctahydro-1H-pyrazino[2,1-c][1,2,4]triazine-1-carboxamide,
(6S,9S)—N-benzyl-6-(4-hydroxybenzyl)-9-methyl-8-(naphthalen-1-ylmethyl)-4,7-dioxohexahydropyrazino[2,1-c][1,2,4]oxadiazine-1(6H)-carboxamide,
(6S,9S)-8-((2-aminobenzo[d]thiazol-4-yl)methyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide,
(6S,9S)-2-allyl-N-benzyl-6-(4-hydroxybenzyl)-9-methyl-4,7-dioxo-8-(quinolin-8-ylmethyl)octahydro-1H-pyrazino[2,1-c][1,2,4]triazine-1-carboxamide,
4-(((6S,9S)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate,
4-(((6S,9S)-1-(benzylcarbamoyl)-2,9-dimethyl-8-(naphthalen-1-ylmethyl)-4,7-dioxooctahydro-1H-pyrazino[2,1-c][1,2,4]triazin-6-yl)methyl)phenyl dihydrogen phosphate,
sodium 4-(((6S,9S)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl phosphate,
sodium 4-(((6S,9S)-1-(benzylcarbamoyl)-2,9-dimethyl-4,7-dioxo-8-(naphthalen-8-ylmethyl)octahydro-1H-pyrazino[2,1-c][1,2,4]triazin-6-yl)methyl)phenyl phosphate,
(6S,9S)-2-allyl-6-(4-hydroxybenzyl)-9-methyl-4,7-dioxo-N—((R)-1-phenylethyl)-8-(quinolin-8-ylmethyl)octahydro-1H-pyrazino[2,1-c][1,2,4]triazine-1-carboxamide,
(6S,9S)-2-allyl-6-(4-hydroxybenzyl)-9-methyl-4,7-dioxo-N—((S)-1-phenylethyl)-8-(quinolin-8-ylmethyl)octahydro-1H-pyrazino[2,1-c][1,2,4]triazine-1-carboxamide,
(6S,9S)—N-benzyl-6-(4-hydroxy-2,6-dimethylbenzyl)-2,9-dimethyl-4,7-dioxo-8-(quinolin-8-ylmethyl)octahydro-1H-pyrazino[2,1-c][1,2,4]triazine-1-carboxamide,
(6S,9S)-8-(benzo[b]thiophen-3-ylmethyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)-8-(benzo[c][1,2,5]thiadiazol-4-ylmethyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-8-(isoquinolin-5-ylmethyl)-2,9-dimethyl-4,7-dioxooctahydro-1H-pyrazino[2,1-c][1,2,4]triazine-1-carboxamide,
(6S,9S)—N-benzyl-8-((5-chlorothieno[3,2-b]pyridin-3-yl)methyl)-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-2,9-dimethyl-4,7-dioxo-8-(quinoxalin-5-ylmethyl)octahydro-1H-pyrazino[2,1-c][1,2,4]triazine-1-carboxamide, and
(6S,9S)-6-(4-hydroxybenzyl)-2,9-dimethyl-4,7-dioxo-8-(quinolin-8-ylmethyl)-N-(thiophen-2-ylmethyl)octahydro-1H-pyrazino[2,1-c][1,2,4]triazine-1-carboxamide.
   These compounds are especially useful in the prevention and/or treatment of viral and infectious diseases, including HIV, HPV, HBV, HSV, and bacteria including Mycobacterium, Shigella, and Listeria.

In a most preferred embodiment, the compound is:
4-(((6S,9S,9aS)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate (Compound A), or
(6S,9S,9aS)-N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide (Compound C).
   These compounds are especially useful in the prevention and/or treatment of viral and infectious diseases, including HIV, HPV, HBV, HSV, and bacteria including Mycobacterium, Shigella, and Listeria.

While not wishing to be bound, the effectiveness of these compounds in treating these conditions is based in part on the ability of these compounds to block TCF4/β-catenin transcriptional pathway by inhibiting cyclic AMP response-element binding protein (CBP), thus altering wnt pathway signaling, which has been found to improve outcomes.

A “β-catenin inhibitor” is a substance that can reduce or prevent β-catenin activity. β-catenin activities include translocation to the nucleus, binding with TCF (T cell factor) transcription factors, and coactivating TCF transcription factor-induced transcription of TCF target genes. A “β-catenin inhibitor” can also interfere with the interaction of CBP and β-catenin. Thus, a β-catenin inhibitor inhibits or reduces CBP/β-catenin signaling and activity of the CBP/β-catenin signaling pathway, including reduction of one or more downstream signaling events.

Disclosed herein are alpha helix mimetic β-catenin inhibitor compounds for treatment of viral and infectious diseases, including HIV, HPV, HBV, HSV, and bacteria including Mycobacterium, Shigella, and Listeria.

Infectious diseases are diseases caused by invasion of a microorganism such as a virus or bacterium. Examples of infectious diseases treatable by the compounds and methods of the invention are as follows.

Human immunodeficiency virus (HIV) causes acquired immunodeficiency syndrome (AIDS), a condition that causes progressive failure of the immune system and allows opportunistic infections and cancers to grow in the immune-compromised subject.

Hepatitis B is an infectious inflammatory illness of the liver caused by the hepatitis B virus (HBV). The infection is often asymptomatic, but chronic infection can lead to scarring of the liver and ultimately to cirrhosis, which is generally apparent after many years. In some cases, those with cirrhosis will go on to develop liver failure, liver cancer or life-threatening esophageal and gastric varices.

Human papilloma virus (HPV) is a papillomavirus that infects keratinocytes of the skin or mucous membranes. Some types can cause genital warts (verrucae), while others can lead to cancers of the cervix, vulva, vagina, penis, oropharynx and anus. HPV infection is a prevalent cause of cervical dysplasia, a precancerous condition that can progress to cervical cancer if untreated.

Herpes simplex virus 1 and 2 (HSV-1 and HSV-2), also known as Human herpes virus 1 and 2 (HHV-1 and -2), are two members of the herpes virus family. Both HSV-1 (which produces most cold sores) and HSV-2 (which produces most genital herpes) are ubiquitous and contagious. They can be spread when an infected person is producing and shedding the virus. Symptoms of herpes simplex virus infection include watery blisters in the skin or mucous membranes of the mouth, lips or genitals.

Tuberculosis, MTB, or TB (short for tubercle bacillus) is a common, and in many cases lethal, infectious disease caused by various strains of mycobacteria, usually Mycobacterium tuberculosis. Tuberculosis typically attacks the lungs but can also affect other parts of the body. It is spread through the air when people who have an active TB infection cough, sneeze, or otherwise transmit their saliva through the air.

Listeria is a virulent food-borne pathogen that infects multiple cell types including phagocytic cells such as macrophages. L. monocytogenes kills 20 to 30% of individuals with clinical infections, and is a leading cause of meningitis in newborns. Shigella species, particularly S. flexneri, S. sonnei, and S. dysenteriae, infect cells of the gastrointestinal tract and cause severe gastrointestinal symptoms such as diarrhea and stomach cramps. Shigella infections are responsible for over 90 million cases of dysentery and over 100,000 deaths each year, mostly in children in developing countries.

Additional infectious diseases that can be treated by the β-catenin inhibitors of the invention include infections caused by Brucella abortus, Chlamydia trachomatis, Legionella pneumophila, Porphyromonas gingivalis, Salmonella species, Staphylococcus aureus, Streptococcus pyogenes, Coxsackievirus, Cytomegalovirus, Dengue virus, Influenza A virus, Poliovirus, Respiratory syncytial virus, and Varicella zoster virus.

As used herein, “treatment” refers to clinical intervention in an attempt to alter the disease course of the individual or cell being treated, and can be performed during the course of clinical pathology. Therapeutic effects of treatment include without limitation, preventing recurrence of disease, alleviation of symptoms, diminishment of any direct or indirect pathological consequences of the disease, decreasing the rate of disease progression, amelioration or palliation of the disease state, and remission or improved prognosis.

As used herein, the terms “therapeutically effective amount” and “effective amount” are used interchangeably to refer to an amount of a composition of the invention that is sufficient to result in the prevention of the development or onset of viral and infectious diseases, including HIV, HPV, HBV, HSV, and bacteria including Mycobacterium, Shigella, and Listeria, or one or more symptoms thereof, to enhance or improve the effect(s) of another therapy, and/or to ameliorate one or more symptoms of such diseases.

A therapeutically effective amount can be administered to a patient in one or more doses sufficient to palliate, ameliorate, stabilize, reverse or slow the progression of the disease, or otherwise reduce the pathological consequences of the disease, or reduce the symptoms of the disease. The amelioration or reduction need not be permanent, but may be for a period of time ranging from at least one hour, at least one day, or at least one week or more. The effective amount is generally determined by the physician on a case-by-case basis and is within the skill of one in the art. Several factors are typically taken into account when determining an appropriate dosage to achieve an effective amount. These factors include age, sex and weight of the patient, the condition being treated, the severity of the condition, as well as the route of administration, dosage form and regimen and the desired result.

As used herein, the terms “subject” and “patient” are used interchangeably and refer to an animal, preferably a mammal such as a non-primate (e.g., cows, pigs, horses, cats, dogs, rats etc.) and a primate (e.g., monkey and human), and most preferably a human.

The alpha helix mimetic β-catenin inhibitors described herein are useful to prevent or treat disease. Specifically, the disclosure provides for both prophylactic and therapeutic methods of treating a subject at risk of (or susceptible to) viral infection. Accordingly, the present methods provide for the prevention and/or treatment of viral and infectious diseases, including HIV, HPV, HBV, HSV, and bacteria including Mycobacterium, Shigella, and Listeria in a subject by administering an effective amount of the alpha helix mimetic β-catenin inhibitors to a subject in need thereof. For example, a subject can be administered the alpha helix mimetic β-catenin inhibitors in an effort to improve one or more of the symptoms of a viral infection.

Inhibition of Wnt/beta catenin has the potential to modify cells such that they become a less optimal host for the virus. Accordingly, the β-catenin inhibitors of the invention can be given in combination with an antiviral compound that would become more effective in the setting of the modified host cell.

Thus, the invention encompasses methods where the compound is given in combination therapy. That is, the compound can be used in conjunction with, but separately from, other agents useful in treating viral or bacterial infection. In these combination methods, the compound will generally be given in a daily dose of 1-100 mg/kg body weight daily in conjunction with other agents. The other agents generally will be given in the amounts used therapeutically. The specific dosing regime, however, will be determined by a physician using sound medical judgment.

Some examples of compounds suitable for compositions and methods involving antiviral combination therapy with the inhibitory compounds disclosed herein include, but are not limited to, the following: antiviral agents such as acyclovir and its prodrug valacyclovir; ganciclovir and its prodrug valganciclovir; foscavir; brivudin; cidofovir; adefovir; lamivudine; boceprevir; entecavir; genital wart topical treatments such as imiquimod, podofilox, or cryosurgery; pegylated interferons; reverse transcriptase inhibitors; protease inhibitors; HIV integrase strand transfer inhibitors; HIV fusion and entry inhibitors; and histone deacetylase complex (HDAC) inhibitors. HDAC inhibitors include, but are not limited to, hydroxamic acids (or hydroxamates) such as trichostatin A, vorinostat (SAHA), abexinostat (PCI-24781), belinostat (PXD101), LAQ824, and panobinostat (LBH589); PCI-34051; cyclic tetrapeptides such as trapoxin B; depsipeptides such as romidepsin; benzamides such as entinostat (MS-275), CI994, and mocetinostat (MGCD0103); electrophilic ketones; aliphatic acid compounds such as phenylbutyrate and valproic acid; nicotinamides, and NAD derivatives such as dihydrocoumarin, naphthopyranone, and 2-hydroxynaphaldehydes.

Combination therapy with the inhibitory compounds disclosed herein for bacterial infections such as infection by Mycobacterium, Shigella, and Listeria include, but are not limited to, the following: isoniazid, rifampin, rifapentine, ethambutol, and pyrazinamide.

Treatment of infectious diseases, including HIV, HPV, HBV, HSV, and bacteria including Mycobacterium, Shigella, and Listeria, refers to the administration of a compound or combination described herein to treat a subject suffering from such an infectious disease. One outcome of the treatment of infectious disease is to reduce symptoms of the disease. Another outcome of the treatment of infectious disease is to reduce inflammation and infiltration of immune cells. Still another outcome of the treatment of infectious disease is to reduce infiltration of the microorganism into the host cells or tissues. Still another outcome of the treatment of infectious disease is to reduce spread of the microorganism causing the infection.

The alpha helix mimetic β-catenin inhibitors described herein can be incorporated into pharmaceutical compositions for administration, singly or in combination, to a subject for the treatment or prevention of a disorder described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Any suitable route of administration may be employed for providing a mammal, especially a human, with an effective dose of a compound described herein. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The effective dosage of active ingredient employed may vary depending on the particular compound employed, the mode of administration, the condition being treated and the severity of the condition being treated. Such dosage may be ascertained readily by a person skilled in the art.

When treating or controlling infectious diseases, including HIV, HPV, HBV, HSV, and tuberculosis, generally satisfactory results are obtained when the compounds described herein are administered at a daily dosage of from about 0.01 milligram to about 100 milligram per kilogram of animal body weight, preferably given as a single daily dose or in divided doses two to six times a day, or in sustained release form. For most large mammals, the total daily dosage is from about 1.0 milligrams to about 1000 milligrams. In the case of a 70 kg adult human, the total daily dose will generally be from about 1 milligram to about 500 milligrams. For a particularly potent compound, the dosage for an adult human may be as low as 0.1 mg. In some cases, the daily dose may be as high as 1 gram. The dosage regimen may be adjusted within this range or even outside of this range to provide the optimal therapeutic response.

Oral administration will usually be carried out using tablets or capsules. Examples of doses in tablets and capsules are 0.1 mg, 0.25 mg, 0.5 mg, 1 mg, 2 mg, 5 mg, 10 mg, 15 mg, 20 mg, 25 mg, 30 mg, 40 mg, 50 mg, 100 mg, 200 mg, 250 mg, 300 mg, 400 mg, 500 mg, and 750 mg. Other oral forms may also have the same or similar dosages.

Also described herein are pharmaceutical compositions which comprise a compound described herein and a pharmaceutically acceptable carrier. The pharmaceutical compositions described herein comprise a compound described herein or a pharmaceutically acceptable salt as an active ingredient, as well as a pharmaceutically acceptable carrier and optionally other therapeutic ingredients. A pharmaceutical composition may also comprise a prodrug, or a pharmaceutically acceptable salt thereof, if a prodrug is administered.

The compositions can be suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds described herein can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions as oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds described herein may also be administered parenterally. Solutions or suspensions of these active compounds can be prepared in water suitably mixed with a surfactant or mixture of surfactants such as hydroxypropylcellulose, polysorbate 80, and mono and diglycerides of medium and long chain fatty acids. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

### EXAMPLES

The methods for the EXAMPLES of this study are as follows.

Cell Sorting and Flow Cytometry—

PBMC were stained with monoclonal antibodies directed against CD4, CD3, CD45RA, CCR7, CD62L, CD122, CD95, according to standard protocols. After 20 minutes, CCR7+ CD45RA+ naïve CD4 T cells, CCR7+ CD45RA− central-memory CD4 T cells (CD4 Tcm), CCR7− CD45RA− CD4 T cells, CCR7− CD45RA+ terminally-differentiated CD4 T cells (CD4 Ttd) and CCR7+ CD45RA+ CD62L+ CD95+ CD122+ T memory stem cell CD4 T cells (CD4 Tscm) were live sorted in a specifically designated biosafety cabinet (Baker Hood), using a FACS Aria cell sorter (BD Biosciences) at 70 pounds per square inch. For phenotypic characterization, cells were additionally stained with CCR5 or CXCR4 antibodies, or Annexin V, and acquired on a LSRII flow cytometer (BD Biosciences). Data were analyzed using FlowJo software (Treestar).

Assessment of Cell-Associated HIV-1 DNA—

Isolated CD4 T cells were digested as previously described (Nat Med 15, 893-900 (2009)) to extract cell lysates. The inventors amplified total HIV-1 DNA with primers and probes previously described (Methods 47, 254-260 (2009)). As a standard curve, the inventors amplified serial dilutions of chronically infected 293T cells (kindly provided by Dr. Bushman, University of Pennsylvania). Proviral HIV-1 DNA copy numbers were calculated relative to CCR5 gene copy number previously quantified with the same standards.

Analysis of Cell-Associated Unspliced HIV-1 RNA—

Cell-associated unspliced HIV-1 RNA in sorted CD4 T cells were quantified by seminested real-time PCR, using a previously described protocol (J Infect Dis 206, 1443-1452 (2012)). Results were calculated as the number of HIV-1 RNA copies per microgram of total RNA. Samples from all patients were processed together to avoid inter-assay variability.

In Vitro Infection Assays—

Unselected PBMC from HIV-1 negative donors without prior in vitro activation were cultured in RPMI medium supplemented with 10% FCS and 50 U/ml of rhIL-2. A total of 10×10⁶ PBMCs were infected with a GFP-encoding VSV-G-pseudotyped virus (MOI=0.01) or a GFP-encoding R5-tropic viral strains (Ba-L, MOI=0.07, kindly provided by Dr. Littman, New York University). Cells were then washed twice with PBS and cultured at 10⁶ cells/ml in 96 round-bottom well plates for 5 days. On day 5, cells were stained with surface antibodies to identify individual CD4 T cell subsets, washed and analyzed on a LSRII flow cytometer instrument.

Analysis of HIV-1 Replication Products—

HIV-1 late reverse transcripts were amplified from cell lysates with primers MH531 and MH532 and probe LRT-P, as previously described. Integrated HIV-1 DNA was detected using nested PCR with Alu-1/Alu-2 primers and HIV-1 LTR primer L-M667 for the first-round PCR and LTR primer AA55M, Lambda T primers, and MH603 probe for the second-round quantitative PCR. HIV-1 2-LTR DNA was amplified using an established protocol. Amplification of the housekeeping gene CCR5 was used to quantify input cell numbers. Serial dilutions of DNA from cell lysates of the HIV-1-infected cell line 293T (provided by F. Bushman, University of Pennsylvania, Philadelphia, Pa., USA) were used for reference purposes.

Viral Outgrowth Assays—

Viral outgrowth assays were performed as previously described with some modifications (Methods Mol Biol 304, 3-15 (2005)). Sorted CD4⁺ T cell populations were seeded at 10,000 cells/well (Tscm) or 20,000 cells/well (Tcm and Tem) in round-bottom 96-well plates. Subsequently, cells were stimulated with PHA (2 mg/ml), rh IL-2 (100 units/ml) and irradiated allogeneic PBMCs from HIV-negative healthy donors. CD8-depleted, PHA-stimulated PBMC from HIV-negative donors were added to each well on day 3 and again on day 7 and 14 of culture. The cultures were subjected to removal of 33% of the cell suspension every seven days and replenished with fresh rh IL-2 containing (100 U/ml) media. After 14-21 days, cell supernatant from each well was harvested and the number of wells containing infectious HIV-1 was assessed by incubation of the supernatant with TZM-bl cells, a permissive HeLa cell clone that contains integrated reporter genes for firefly luciferase under control of an HIV-1 LTR, permitting sensitive and accurate measurements of infection. Luciferase activity was quantified by luminescence and is directly proportional to the number of infectious virus particles present in the initial inoculum.

Viral Sequencing—

Cell lysates from sorted T cell populations and plasma were used for HIV-1 envelope sequencing encompassing the V3 region. For plasma samples, a median of 6 mL of plasma from each time point were ultracentrifuged at 170.000 g for 30 min prior to proteinase K digestion and RNA isolation by acid guanidinium isothiocyanate. One-step RT-PCR reaction (Superscript III, Invitrogen) was performed in triplicates using outer primers envA/LA17 (PLoS Pathog 7, e1001303 (2011)). Pooled PCR products were used as a template to generate a single amplicon by nested PCR with inner primers LA12 and LA13. Amplification products were inserted into TOPO cloning vectors, and used to transform competent bacteria. Individual bacterial colonies were amplified by overnight culture, and extracted DNA was ligated and directly sequenced by T7 or T3 primers on an ABI 3100 PRISM automated sequencer, without prior PCR-based amplification. jModeltest v0.1.1 was used to infer the best phylogenetic model to explain the alignment sequence evolution.

In Vitro Culture Assays—

Selected CD4 T cell subsets were isolated by cell sorting, labeled cells with 2 μM CFSE for 7 min at 37° C., and incubated with rhIL-15 (25 ng/ml; Peprotech) for 10 d in the presence or absence of Compound C, (6S,9S,9aS)-N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide (Prism Biolabs). Afterwards, cells were harvested and phenotypically characterized by flow cytometry.

Statistics—

Data are summarized as means and SD or using box and whisker plots (indicating the median, interquartile range, and minimum and maximum values). Pearson's correlation coefficient was calculated to analyze correlations. Differences between nominal data were tested for statistical significance by 2-tailed Student's t test, Mann-Whitney U test, or paired Wilcoxon rank-sum.

### Example 1

### Compound C Inhibits HIV in a Dose Dependent Manner

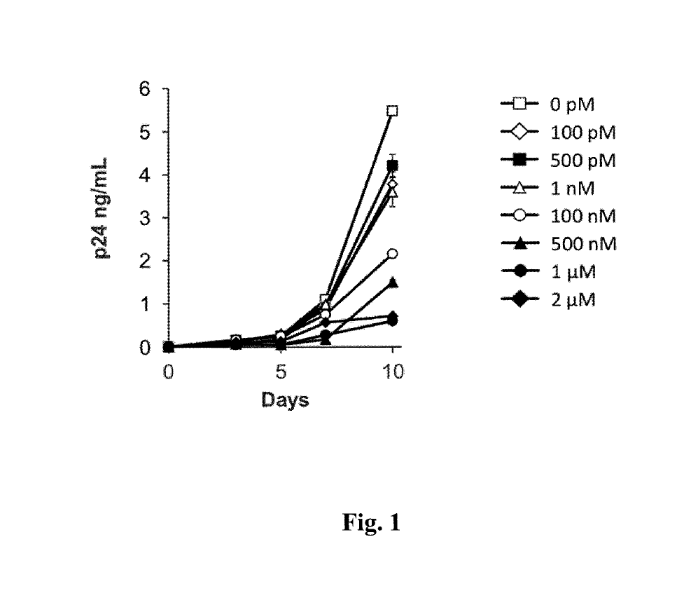

For these experiments, peripheral blood mononuclear cells (PBMCs) were isolated from HIV seronegative donors and differentiated into macrophages for 7-10 days using methods previously described (J Biol Chem 286: 18890-18902 (2011); PLoS Pathogens 8(5):e1002689 pp 1-13 (2012); PLoS Pathogens 8(11):e1003017 (2012)). The macrophages were pretreated for 24 hours with Compound C, which is the β-catenin inhibitor (6S,9S,9aS)-N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide. Macrophages were treated with Compound C at increasing concentrations and productive infection was monitored for 10 days by detection of HIV p24 antigen released into the culture supernatants. As shown in FIG. 1, Compound C inhibits HIV replication in a dose-dependent manner.

Conclusion: Compound C inhibits HIV in a dose dependent manner at concentrations ranging from 100 pM to 2 μM.

### Example 2

### Compound A at 1 and 2 μM Induces Autophagy

During permissive infection, HIV down-regulates autophagy. Rapamycin, an inhibitor of mTOR, and 1α,25-dihydroxycholecalciferol (1,25D3), the hormonally active form of vitamin D3, exert anti-Mtb and anti-HIV activity in human macrophages through macroautophagy (autophagy). The hallmark of autophagy is a double-membraned autophagosome that engulfs bulk cytoplasm and cytoplasmic organelles such as mitochondria and endoplasmic reticulum. Autophagosomes ultimately fuse with lysosomes thereby generating single-membraned autolysosomes that are capable of degrading the contents which can then be recycled by the cell. Autophagy has been recognized as an efficient mechanism of innate immunity against certain bacteria, viruses and other pathogens (sometimes termed xenophagy).

Microtubule-associated protein 1A/1B-light chain 3 (LC3) is a soluble protein that is distributed ubiquitously in mammalian tissues and cultured cells (reviewed in Methods Mol. Biol. 445, 77-88 (2008). During autophagy, autophagosomes engulf cytoplasmic components, including cytosolic proteins and organelles. Concomitantly, a cytosolic form of LC3 (LC3-I) is conjugated to phosphatidylethanolamine to form LC3-phosphatidylethanolamine conjugate (LC3-II), which is recruited to autophagosomal membranes. Autophagosomes fuse with lysosomes to form autolysosomes, and intra-autophagosomal components are degraded by lysosomal hydrolases. At the same time, LC3-II in autolysosomal lumen is degraded. Thus, lysosomal turnover of the autophagosomal marker LC3-II reflects starvation-induced autophagic activity, and detecting LC3 by immunoblotting or immunofluorescence is an indicator of autophagy and autophagy-related processes, including autophagic cell death.

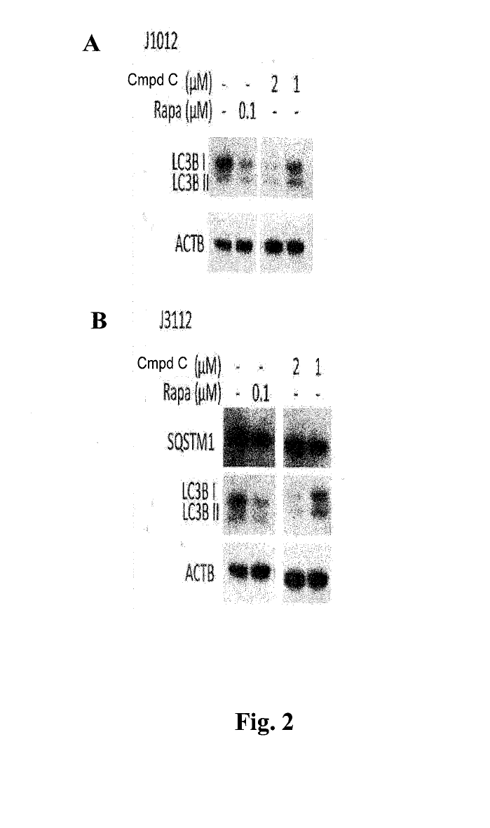

FIG. 2 shows experiments performed to assess if Compound A, which is the β-catenin inhibitor 4-(((6S,9S,9aS)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate, induces microtubule-associated protein 1A/1B-light chain 3B (LC3B, an indicator of autophagy). For these studies, macrophages were exposed to rapamycin 100 nM (a known inducer of autophagic flux), proteins were extracted and subjected to immunoblotting.

During autophagy, LC3B (LC3B-I) forms LC3B-phosphatidylethanolamine conjugate (LC3B-II), which is recruited to autophagosomal membranes. When there is autophagic flux there is lipidation of LC3B-I that results in conversion of LC3B-II. Thus, during autophagic flux the relative quantity of LC3B-II is increased compared to LC3B-I.

As seen in FIGS. 2A-2B, the ratio of LC3B-II to LC3B-I is increased relative to negative/untreated controls, indicating increased autophagy.

In addition, the inventors looked at another marker of autophagic flux (sequestosome 1, also called SQSTIM1 or p62). During active autophagy, SQSTIM1 is degraded (decreased). As seen in FIG. 2B, SQSTIM1 decreases with β-catenin inhibitor at 1 and 2 μM as well as with the rapamycin control.

Importantly, at the concentrations of β-catenin inhibitor studied, to date, no cell toxicity has been observed.

Conclusion: Compound A at 1 and 2 μM induces autophagy, and thus can improve immunity against HIV and Mtb infection.

### Example 3

### Targeting HIV Reservoir Cells with β-Catenin Inhibitors

Latently infected CD4 T cells represent a transcriptionally silent reservoir for HIV-1 and harbor chromosomally integrated viral DNA capable of resuming HIV-1 replication upon activation and antiretroviral treatment discontinuation (Science 278, 1295-1300 (1997); Nature Medicine 5, 512-517 (1999)). These cells primarily consist of long-lived memory T cells with a slow spontaneous decay rate, suggesting that HIV-1 exploits physiologic mechanisms of cellular immune memory for promoting viral persistence (Nat Med 15, 893-900 (2009)). Recently, small proportions of T cells with stem cell characteristics have been discovered in some animal species. These cells, termed “T memory stem cells” (Tscm), seem to represent the earliest developmental stage of memory T cells. Functionally, Tscm exceed the proliferative capacity of all known alternative T cell subsets, and can differentiate into large numbers of central-memory (Tcm), effector-memory (Tem) and terminally-differentiated T (Ttd) cells.

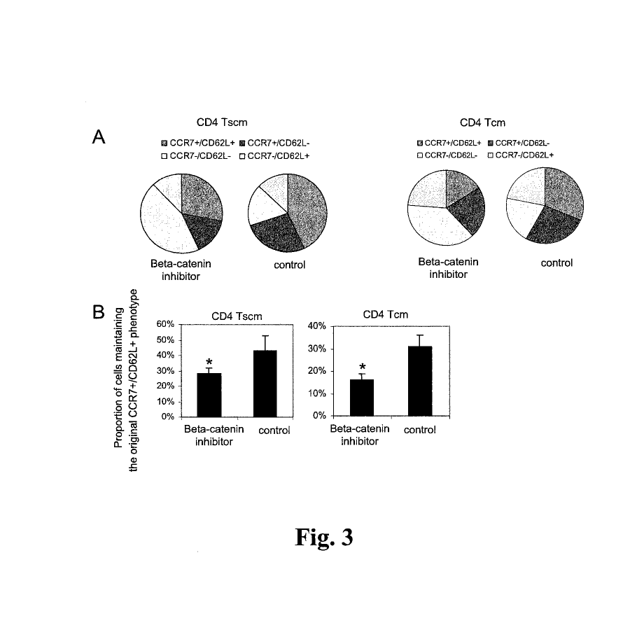

To evaluate the effects of β-catenin inhibitors on CD4 T cell development, sorted Tscm and Tcm were incubated with Compound C, which is the β-catenin inhibitor (6S,9S,9aS)-N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide. Compound C is the active metabolite of Compound A. The inventors observed that in comparison to control experiments, Compound C substantially facilitated differentiation of Tscm, and to a lesser extent, of Tcm into more mature, CCR7− CD62L− negative CD4 T cell populations (FIGS. 3A-3B).

Conclusion: treatment of HIV-infected cells by the β-catenin inhibitors disclosed herein facilitates differentiation of HIV-1 infected Tscm into more mature, short-lived T cells with reduced in vivo persistence. This can reduce the reservoir of HIV-infected cells and thus lead to eradication of the virus.

1. An alpha helix mimetic β-catenin inhibitor compound for the treatment of viral and infectious diseases, having the following formula (I): 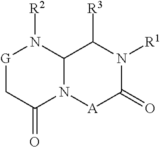
wherein:
A is —CHR⁷—,
wherein R⁷ is hydrogen, optionally substituted alkyl, optionally substituted alkenyl, optionally substituted alkynyl, optionally substituted arylalkyl, optionally substituted heteroarylalkyl, optionally substituted cycloalkylalkyl, optionally substituted heterocycloalkylalkyl, optionally substituted aryl, optionally substituted heteroaryl, optionally substituted cycloalkyl or optionally substituted heterocycloalkyl;
G is —NH—, —NR⁶—, —O—, —CHR⁶— or —C(R⁶)₂—,
wherein R⁶ is independently selected from optionally substituted alkyl, optionally substituted alkenyl and optionally substituted alkynyl;
R¹ is optionally substituted arylalkyl, optionally substituted heteroarylalkyl, optionally substituted cycloalkylalkyl or optionally substituted heterocycloalkylalkyl;
R² is —W²¹—W²²—Rb—R²⁰,
wherein W²¹ is —(CO)— or —(SO₂)—; W²² is bond, —O—, —NH— or optionally substituted lower alkylene; Rb is bond or optionally substituted lower alkylene; and R²⁰ is optionally substituted alkyl, optionally substituted alkenyl, optionally substituted alkynyl, optionally substituted aryl, optionally substituted heteroaryl, optionally substituted cycloalkyl or optionally substituted heterocycloalkyl; and
R³ is optionally substituted alkyl, optionally substituted alkenyl or optionally substituted alkynyl;
or a pharmaceutically acceptable salt thereof.

2. The compound of claim 1, selected from:
(6S,9S)—N-benzyl-6-(4-hydroxybenzyl)-2,9-dimethyl-8-(naphthalen-1-ylmethyl)-4,7-dioxooctahydro-1H-pyrazino[2,1-c][1,2,4]triazine-1-carboxamide,
(6S,9S)-2-allyl-N-benzyl-6-(4-hydroxybenzyl)-9-methyl-8-(naphthalen-1-ylmethyl)-4,7-dioxooctahydro-1H-pyrazino[2,1-c][1,2,4]triazine-1-carboxamide,
(6S,9S)—N-benzyl-6-(4-hydroxybenzyl)-9-methyl-8-(naphthalen-1-ylmethyl)-4,7-dioxohexahydropyrazino[2,1-c][1,2,4]oxadiazine-1 (6H)-carboxamide,
(6S,9S)-8-((2-aminobenzo[d]thiazol-4-yl)methyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide,
(6S,9S)-2-allyl-N-benzyl-6-(4-hydroxybenzyl)-9-methyl-4,7-dioxo-8-(quinolin-8-ylmethyl)octahydro-1H-pyrazino[2,1-c][1,2,4]triazine-1-carboxamide,
4-(((6S,9S)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate,
4-(((6S,9S)-1-(benzylcarbamoyl)-2,9-dimethyl-8-(naphthalen-1-ylmethyl)-4,7-dioxooctahydro-1H-pyrazino[2,1-c][1,2,4]triazin-6-yl)methyl)phenyl dihydrogen phosphate,
sodium 4-(((6S,9S)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl phosphate,
sodium 4-(((6S,9S)-1-(benzylcarbamoyl)-2,9-dimethyl-4,7-dioxo-8-(naphthalen-8-ylmethyl)octahydro-1H-pyrazino[2,1-c][1,2,4]triazin-6-yl)methyl)phenyl phosphate,
(6S,9S)-2-allyl-6-(4-hydroxybenzyl)-9-methyl-4,7-dioxo-N—((R)-1-phenylethyl)-8-(quinolin-8-ylmethyl)octahydro-1H-pyrazino[2,1-c][1,2,4]triazine-1-carboxamide,
(6S,9S)-2-allyl-6-(4-hydroxybenzyl)-9-methyl-4,7-dioxo-N—((S)-1-phenylethyl)-8-(quinolin-8-ylmethyl)octahydro-1H-pyrazino[2,1-c][1,2,4]triazine-1-carboxamide,
(6S,9S)—N-benzyl-6-(4-hydroxy-2,6-dimethylbenzyl)-2,9-dimethyl-4,7-dioxo-8-(quinolin-8-ylmethyl)octahydro-1H-pyrazino[2,1-c][1,2,4]triazine-1-carboxamide,
(6S,9S)-8-(benzo[b]thiophen-3-ylmethyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)-8-(benzo[c][1,2,5]thiadiazol-4-ylmethyl)-N-benzyl-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-8-(isoquinolin-5-ylmethyl)-2,9-dimethyl-4,7-dioxooctahydro-1H-pyrazino[2,1-c][1,2,4]triazine-1-carboxamide,
(6S,9S)—N-benzyl-8((5-chlorothieno[3,2-b]pyridin-3-yl)methyl)-6-(4-hydroxybenzyl)-2,9-dimethyl-4,7-dioxooctahydro-1H-pyrazino[2,1-c][1,2,4]triazine-1-carboxamide,
(6S,9S)—N-benzyl-6-(4-hydroxybenzyl)-2,9-dimethyl-4,7-dioxo-8-(quinoxalin-5-ylmethyl)octahydro-1H-pyrazino[2,1-c][1,2,4]triazine-1-carboxamide, and
(6S,9S)-6-(4-hydroxybenzyl)-2,9-dimethyl-4,7-dioxo-8-(quinolin-8-ylmethyl)-N-(thiophen-2-ylmethyl)octahydro-1H-pyrazino[2,1-c][1,2,4]triazine-1-carboxamide.

3. The compound of claim 1, selected from:
4-(((6S,9S,9aS)-1-(benzylcarbamoyl)-2,9-dimethyl-4,7-dioxo-8-(quinolin-8-ylmethyl) octahydro-1H-pyrazino[2,1-c][1,2,4]triazin-6-yl)methyl)phenyl dihydrogen phosphate, and
(6S,9S,9aS)-N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide.

4. A pharmaceutical composition comprising the compound of claim 1.

5. A method of treatment for infectious disease, comprising administering an effective amount of the compound of claim 1 to a patient in need thereof.

6. The method of claim 5, wherein the infectious disease is human immunodeficiency virus (HIV) infection.

7. The method of claim 5, wherein the infectious disease is hepatitis B virus (HBV) infection.

8. The method of claim 5, wherein the infectious disease is tuberculosis (TB).

9. The method of claim 5, wherein the infectious disease is herpes simplex virus 1 (HSV-1) infection.

10. The method of claim 5, wherein the infectious disease is herpes simplex virus 2 (HSV-2) infection.

11. The method of claim 5, wherein the infectious disease is human papilloma virus (HPV) infection.

12. The method of claim 11, wherein the method treats or prevents cervical dysplasia.

13. The method of claim 11, wherein the method treats or prevents genital warts.

14. The method of claim 5, wherein the infectious disease is infection by Shigella or Listeria species.

15. The method of claim 5, further comprising administration of one or more antiviral agents selected from acyclovir, valacyclovir, ganciclovir, valganciclovir, foscavir, brivudin, cidofovir, adefovir, lamivudine, boceprevir, entecavir, imiquimod, podofilox, a pegylated interferon, a reverse transcriptase inhibitor, a protease inhibitor, an HIV integrase strand transfer inhibitor, an HIV fusion or HIV entry inhibitor; and a histone deacetylase complex (HDAC) inhibitor.

16. The method of claim 15, wherein one of the one or more antiviral agents is an HDAC inhibitor selected from a hydroxamic acid, trichostatin A, vorinostat, abexinostat, belinostat, LAQ824, panobinostat, PCI-34051, a cyclic tetrapeptide, trapoxin B, a depsipeptide, romidepsin, a benzamide, entinostat, CI994, mocetinostat (MGCD0103), an electrophilic ketone; an aliphatic acid compound, phenylbutyrate, valproic acid, a nicotinamide, dihydrocoumarin, naphthopyranone, and a 2-hydroxynaphaldehyde.
